(19) United States
(12) Patent Application Publication
(10) Pub. No.: US 2014/0039261 A1
(43) Pub. Date: Feb. 6, 2014

(54) OPTICAL COHERENCE TOMOGRAPHY SYSTEM AND METHOD FOR REAL-TIME SURGICAL GUIDANCE

(71) Applicant: The Johns Hopkins University, Baltimore, MD (US)

(72) Inventors: Jin Ung Kang, Ellicott City, MD (US); Mingtao Zhao, Baltimore, MD (US); Ishan Ann Tsay, Louisville, CO (US); Russell H. Taylor, Severna Park, MD (US)

(73) Assignee: The Johns Hopkins University, Baltimore, MD (US)

(21) Appl. No.: 13/957,145

(22) Filed: Aug. 1, 2013

Related U.S. Application Data
(60) Provisional application No. 61/678,397, filed on Aug. 1, 2012.

(51) Int. Cl. A61B 19/00

(52) U.S. Cl. CPC A61B 19/20
USPC 600/166; 600/160; 600/178; 606/130

(57) ABSTRACT

An optical coherence tomography (OCT) system for real-time surgical guidance includes an optical source, an optical fiber configured to be optically coupled to the optical source, a plurality of OCT sensor heads configured to be optically coupled to the optical fiber, an optical detector configured to be optically coupled to the optical fiber, a signal processor configured to communicate with the optical detector to receive detected signals therefrom, and a display system configured to receive OCT image signals from the signal processor and to display an OCT image of at least a portion of a surgical region of interest in real time to provide surgical guidance. The plurality of OCT sensor heads includes a bulk sensor head configured to image at least a portion of the surgical region of interest from an external imaging position and an endoscopic sensor head configured to be inserted into the surgical region of interest to image at least a portion of the surgical region of interest from an internal imaging position. The bulk sensor head and the endoscopic sensor head are at least one of separate exchangeable sensor heads or a reconfigurable sensor head.

### CROSS-REFERENCE OF RELATED APPLICATION

This application claims priority to U.S. Provisional Application No. 61/678,397 filed Aug. 1, 2012, the entire contents of which are hereby incorporated by reference.

This invention was made with Government support of Grant No. R01 EY021540, awarded by the Department of Health and Human Services, The National Institutes of Health (NIH). The U.S. Government has certain rights in this invention.

### BACKGROUND 1. Field of Invention

The field of the currently claimed embodiments of this invention relates to optical coherence tomography (OCT) systems, and more particularly to OCT systems real-time surgical guidance.

2. Discussion of Related Art

Optical coherence tomography based sensing and imaging is a highly effective technique for non-destructive cross-sectional imaging of biological tissues[1-5]. Recently it has been demonstrated that OCT can be highly effective in freehand or robotically assisted microsurgery. Another potential application is in cochlear implant surgery to treat patients with hearing issues[6].

Cochlear implant surgery is a difficult procedure involving delicate tissues and highly confined spaces within temporal bone. Precise cochlear implantation requires a reliable knowledge of the cochlea dimensions and the location of the surrounding critical tissues. Thus there remains a need for OCT systems that can provide real-time surgical guidance for cochlear implant surgery.

### SUMMARY

An optical coherence tomography (OCT) system for real-time surgical guidance according to an embodiment of the current invention includes an optical source, an optical fiber configured to be optically coupled to the optical source, a plurality of OCT sensor heads configured to be optically coupled to the optical fiber, an optical detector configured to be optically coupled to the optical fiber, a signal processor configured to communicate with the optical detector to receive detected signals therefrom, and a display system configured to receive OCT image signals from the signal processor and to display an OCT image of at least a portion of a surgical region of interest in real time to provide surgical guidance. The plurality of OCT sensor heads includes a bulk sensor head configured to image at least a portion of the surgical region of interest from an external imaging position and an endoscopic sensor head configured to be inserted into the surgical region of interest to image at least a portion of the surgical region of interest from an internal imaging position. The bulk sensor head and the endoscopic sensor head are at least one of separate exchangeable sensor heads or a reconfigurable sensor head.

A method of performing a procedure using real-time OCT guidance according to an embodiment of the current invention includes obtaining an external OCT image of a region of interest with a bulk sensor head from an external position, at least one of exchanging or reconfiguring the bulk sensor head with an endoscopic senor head, obtaining an internal OCT image of at least a portion of region of interest with the endoscopic sensor head from an internal position, and performing a step of the procedure taking into account information from at least one of the external or internal OCT images.

### BRIEF DESCRIPTION OF THE DRAWINGS

Further objectives and advantages will become apparent from a consideration of the description, drawings, and examples.

FIG. 1 is a schematic illustration of an optical coherence tomography (OCT) system for real-time surgical guidance according to an embodiment of the current invention.

FIGS. 2A-2C are schematic illustrations showing more details of the OCT system of FIG. 1, as follows: A, schematic of one dimensional lensed optical fiber sensing and optical scanning head for two and three dimensional cochlear imaging. B, Dual balanced swept source OCT for two and three-dimensional imaging. C, Non-balanced swept source OCT for cochlear sensing.

FIGS. 3A-3C show A, Sensitivity of CP-OCT is in black (bottom dashed line); experimental results are with error bars; CP-OCT with backward coupling efficiency of 86.5% (1/e² width) is the middle dashed line; traditional balanced SSOCT with backward coupling efficiency of 86.5% is top dashed line. B, retinal layer structure imaging of a cow retina died of 2-hour before imaging, Cochlear canal wall OCT sensing.

FIGS. 4A-4B show basal turn OCT 3D imaging. A, Cross sectional image of basal turn of cochlear. The image size is 2.5 mm (X)×5 mm(Z). B, volume size is 2.5×2.5×5.0 mm. The turn location was clearly showed in the zoom-in region.

FIGS. 5A-5E show results for facial nerve bundle imaging. A, human cadaveric fresh temporal bone with facial recess; B, OCT beam scanning mode; C, cross sectional image of the facial nerve bundle; D, real time 3D volumetric rendering with GPU. Facial fiber bundle shows a band structure; E, cartoon of cochlear.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The term “light” as used herein is intended to have a broad meaning that can include both visible and non-visible regions of the electromagnetic spectrum. For example, visible, near infrared, infrared and ultraviolet light are all considered as being within the broad definition of the term “light.”

Accordingly, an embodiment of the current invention provides a system that can perform both endoscopic and bulk sensing and imaging to assist in cochlear implant surgery. In some embodiments, high-speed OCT can provide sensory and up to 3-D visualization of the cochlea and the surrounding tissues that allows safe and precise execution of the surgical procedure.

The commonly used lensing components in fiber-optic microprobes are gradient-index lenses[7], drum lenses, fiber fused ball lenses, and special liquid-forming ball lenses. For (common-path OCT) CP-OCT cochlear canal sensing, an optimal probe should have a protected reference plane and a smooth, curved, rigid imaging surface with a longer working distance to work properly even if the sensing optical component scratches the stiff bone in cochlear canal. (See, e.g., U.S. patent application Ser. No. 13/709,984 for “SAPPHIRE LENS-BASED OPTICAL FIBER PROBE FOR OPTICAL COHERENCE TOMOGRAPHY” assigned to the same assignee as the current application, the entire contents of which are incorporated herein by reference.) Other main challenges for high speed OCT are tissue-imaging depth in cochlear temporal bone and real time data processing, especially for three-dimensional volumetric rendering. (See also International Application No. PCT/US2011/066603, for “REAL-TIME, THREE-DIMENSIONAL OPTICAL COHERENCE,” assigned to the same assignee as the current application, the entire contents of which are incorporated herein by reference.)

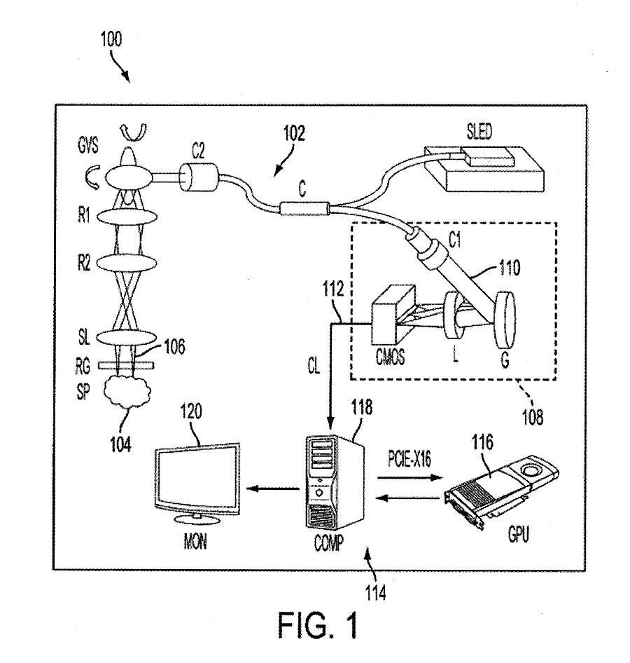

FIG. 1 is a schematic illustration of an optical coherence tomography (OCT) system for real-time surgical guidance 100 according to an embodiment of the current invention. The OCT system 100 includes an optical interferometer 102 configured to illuminate a target 104 with light 106 and to receive light returned from the target 104. The OCT system 100 also includes an optical detector 108 arranged in an optical path of light 110 from the optical interferometer 102 after being returned from the target 104. The optical detector 108 provides detected signals 112. The OCT system 100 further includes a signal processor 114 configured to communicate with the optical detector 108 to receive the detected signals 112. The data processing system 114 can include a parallel processor 116 configured to process the detected signals 112 to provide real-time, three-dimensional optical coherence tomography images of the target 104.

In this example, the optical interferometer 102 includes an SLED as a light source. Other light sources can also be used. The optical interferometer 102 and the optical detector 108 can include further optical components chosen according to the particular application. Some embodiments of the current invention can also include wavelength-swept source based FD-OCT systems.

The parallel processor 116 can be one or more graphics processing units (GPUs) according to an embodiment of the current invention. However, the broad concepts of the current invention are not limited to only embodiments that include GPUs. However, GPUs can provide advantages of cost and speed according to some embodiments of the current invention. In some embodiments, a single GPU can be used. In other embodiments, two or more GPUs can be used. However, the broad concepts of the current invention are not limited to the use of only one or two GPUs. Three, four or more GPUs can be used in other embodiments.

The parallel processor 116 can be installed on a computer 118, for example, but not limited to, with one or more graphics cards. The computer can communicate with the optical detector 108 by direct electrical or optical connections, or by wireless connections, for example. The OCT system 100 can also include one or more display devices, such as monitor 120, as well as any suitable input or output devices depending on the particular application.

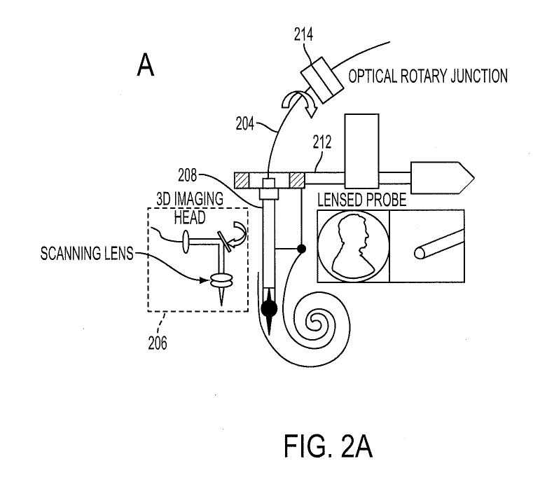

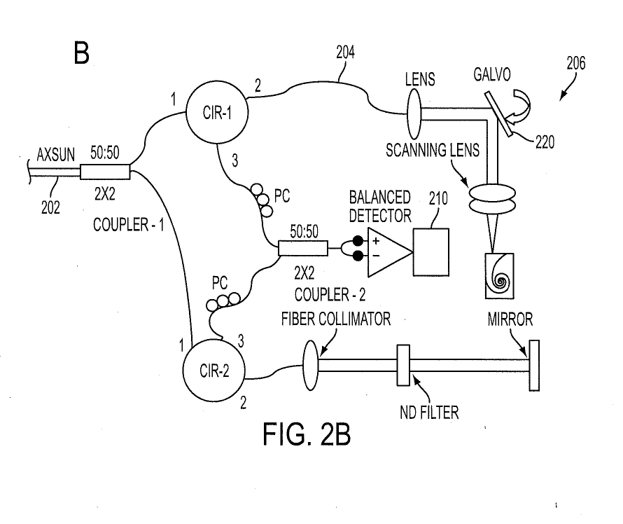

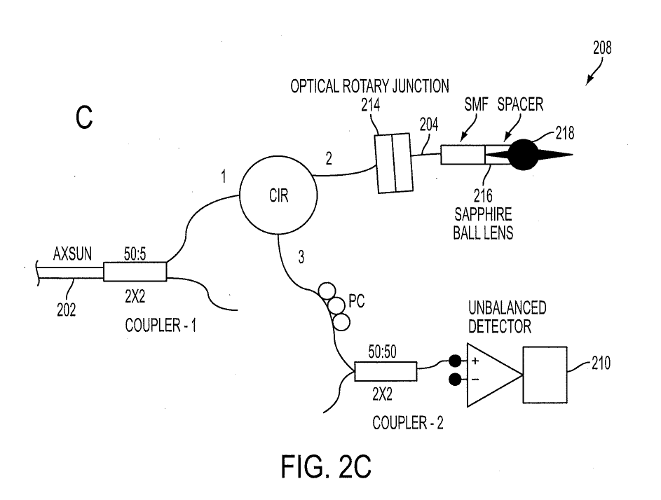

FIGS. 2A-2C provide schematic illustrations of some addition features of the OCT system 100. The OCT system 100 for real-time surgical guidance includes an optical source 202 an optical fiber 204 configured to be optically coupled to the optical source 202, a plurality of OCT sensor heads 206, 208 configured to be optically coupled to the optical fiber 204, an optical detector 210 configured to be optically coupled to the optical fiber 204, a signal processor (see FIG. 1) configured to communicate with the optical detector 210 to receive detected signals therefrom, and a display system (see FIG. 1) configured to receive OCT image signals from the signal processor and to display an OCT image of at least a portion of a surgical region of interest in real time to provide surgical guidance.

The plurality of OCT sensor heads (206, 208) can include a bulk sensor head 206 configured to image at least a portion of the surgical region of interest from an external imaging position and an endoscopic sensor head 208 configured to be inserted into the surgical region of interest to image at least a portion of the surgical region of interest from an internal imaging position. The bulk sensor head 206 and the endoscopic sensor head 208 are at least one of separate exchangeable sensor heads (as shown in FIGS. 2A-2C) or a reconfigurable sensor head. In an embodiment, an endoscopic sensor could be used externally along with an optical adapted attached to the imaging end. After external imaging, the adapter could be removed for continuing with endoscopic imaging. In either case, a frame 212 or other structure of the OCT system 100 can maintain alignment so that endoscopic and bulk imaging remain substantially registered during transition between modes.

Although FIGS. 2A-2C illustrate an embodiment with two OCT sensor heads, the general concepts of the current invention are not limited to only two, There could be three, four, or more sensor heads in other embodiments.

In some embodiments, the OCT system 100 can further include an optical rotary junction 214 configured to be optically coupled to the optical fiber 204 between the optical source and the plurality of OCT sensor heads (206, 208). This can be useful for, but is not limited to, rotating an endoscopic sensor head in which is performing side viewing so as to form a three-dimensional image from within the interior position. The interior position can be created by an incision, and/or a natural cavity or lumen, for example.

In some embodiments, the endoscopic sensor head 208 can include a sheath 216 having a proximal end and a distal end and defining a lumen therein. A sensor optical fiber, which can be the same as or in addition to optical fiber 204, is disposed at least partially within the lumen of the sheath 216. A sapphire lens 218 is attached to the distal end of the sheath to form a fluid-tight seal to prevent fluid from entering the lumen of the sheath 216. The sensor optical fiber 204 has an end arranged in an optical path with the sapphire lens 218 to provide optical coupling between 218 sapphire lens and the sensor optical fiber 204. The sapphire lens 218 can be a substantially spherical sapphire ball lens. The end of said sensor optical fiber 204 can be fixed within the lumen to maintain a predetermined distance from the sapphire lens 218 with a space reserved therebetween.

In some embodiments, the bulk sensor head 206 includes a scanning mirror 220 to scan illumination light, and to receive returned light, across a region to be imaged. In some embodiments, the scanning mirror 220 can be a galvanometer mirror. In some embodiments, the scanning mirror 220 can be a micro-electromechanical system (MEMS) mirror. In some embodiments, the bulk sensor head can be fitted with a borescope having different size and used as an endoscope.

Further additional concepts and embodiments of the current invention will be described by way of the following examples. However, the broad concepts of the current invention are not limited to these particular examples.

### EXAMPLES

Customized Gaussian beam paraxial ray ABCD matrix simulation shows that working distances (WDs) vary with the diameter of the ball lens, wavelengths, and length and type of beam-expanding spacer. Generally, WD at a fixed wavelength is proportional to the diameter of the sapphire ball lens and wavelength. We fabricated two probes in-house to validate the simulation. They were assembled with a single-mode fiber (SMF-28) and a standard 25-gauge hypodermic needle. First, a section of air gap or UV epoxy spacer with refractive index of 1.51 was added between the single-mode fiber distal tip and a sapphire lens with a diameter of 500 μm. Then the air gap or UV epoxy gap were adjusted properly to achieve designed working distance. The reference power is from the fiber distal tip. The WDs were experimentally obtained from the sensitivity falling off of two probes. The parameters of two designs are listed in Table 1.

TABLE 1
Design parameters of two probes (all units in μm)

| Spacer/ | Theoretical | Experimental | DOF/Spot |
|---|---|---|---|
| Length | WD | WD | size |
| Air/275 | 390 | 415 ± 5 | 151/11 |
| UV/169 | 1197 | 1221 ± 15 | 1478/18 |

To the best of our knowledge, no CP-OCT probes have been reported to reach sensitivity up to 88 dB. A dual-balanced detector cannot be used for CP-OCT configurations since it will reject the CP-OCT signal and other common-mode optical noises. To estimate the optimum performance of CP-OCT with an unbalanced detector, we derived the sensitivity model of CP-OCT by modifying the analysis in prior studies. The time-averaged signal power in single port of unbalanced detector of CP-OCT can be expressed as

$$< {i}_{s}^{2} ( t ) >= {( \frac{η e}{hf} )}^{2} {P}_{r} {P}_{s}$$

Here, ^{P}ᵣ and ^{P}ₛ denote the reference and signal power individually; ^{ƒ} is quantum efficiency,^{e} is electron charge,^{h} is Plank's constant. The noise power of a single detector contributed by total noises is given as

$$< {i}_{n}^{2} ( t ) >= [ \frac{4 kT}{R} + \frac{2 η {e}^{2}}{hf} * ( \frac{{P}_{r}}{2} + \frac{{P}_{s}}{2} ) + {( \frac{η e}{hf} )}^{2} * RIN * ( ζ * ( {( \frac{Pr}{2} )}^{2} + {( \frac{Ps}{2} )}^{2} ) + \frac{{P}_{r} {P}_{s}}{2} ) ] * BW$$

where

$$\frac{kT}{4 R}$$

represents thermal noise and the second term is shot noise. The third terms include RIN (relative intensity noise) noise induced by self-beating and cross-beating noises ξ is called the common-mode rejection ratio, which is 0 dB for common-path OCT and typically −35 dB for balanced detector; BW is the bandwidth. Therefore, the sensitivity of the CP-OCT in dB can be expressed as

$$Sensitivity = 10 log ( \frac{{i}_{s}^{2} ( t )}{{i}_{n}^{2} ( t )} )$$

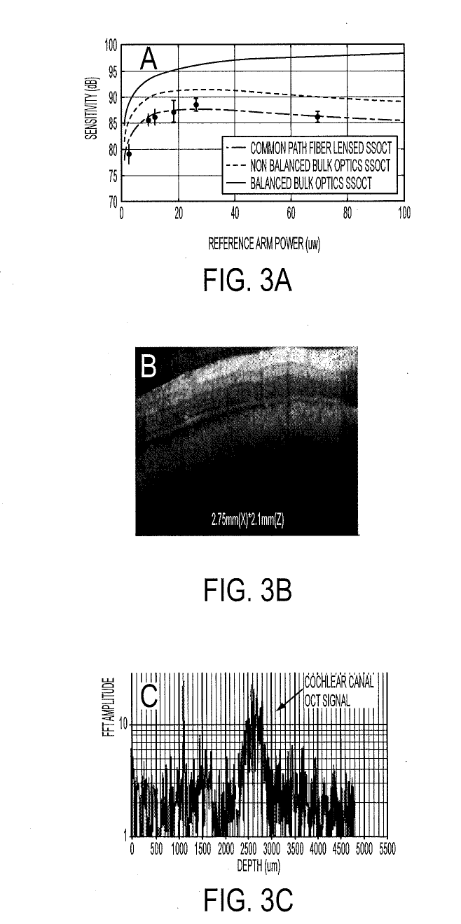

One 25-gauge prototype having common-path fiber probes with lateral resolution of 11 μm has been developed with sensitivity up to 88 dB illustrated in FIG. 3A. The lensed probe could image the phantom of a cow retina over a wide viewing angle. The lensed probe was also used to sense the canal of a dry cadaveric human cochlear temporal bone. The scattering OCT signal can clearly identify the location of the canal wall turning point. The forwarded sensing fiber probe can be replaced with a side view probe driven by an optical rotary junction with rotating speed up to 160 Hz for canal lumen imaging.

In this example we implanted a sapphire ball lens-based fiber-optic CP-OCT probe without complex conjugate issue, with sensitivity up to 88 dB, which works for both spectral domain OCT (SDOCT) and swept source OCT (SSOCT) systems. Swept source OCT at wavelength of 1.3 of a much better imaging depth than spectral domain OCT was used to image cochlear temporal bone. Graphics processing unit (GPU) and C++ were integrated together both for real time two-dimensional cross sectional and three-dimensional volumetric rendering simultaneously. The SSOCT system configuration used is illustrated in FIGS. 2A-2C.

Sapphire ball lenses have excellent optical imaging quality, highly robust, and smooth surface. The high refractive index (n=1.75) of sapphire ball lens allows us to achieve much better lateral resolution of about 10 μm than that of both noncore fiber fused ball lens and Grin lens with around 20-35 μm.

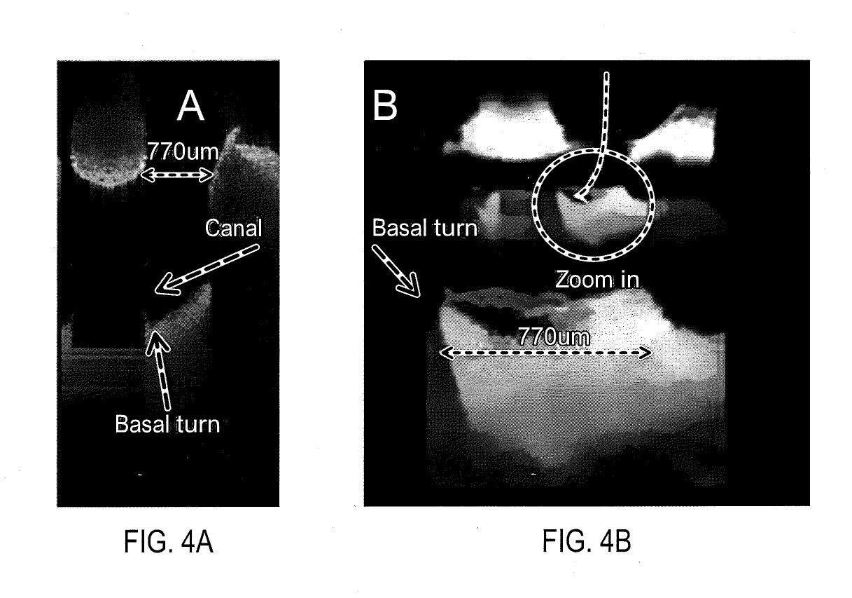

Dual balanced SSOCT was used to scan the basal turn of a cadaveric dry temporal bone, which is important to help surgeon install electrode array during cochlear implant surgery. The scanning optical head is composed of a X-Y Galvo and a telecentric imaging lens with a working distance of 93.7 mm with lateral resolution of 19 μm. We used a swept source laser at 1310 nm with a wide tuning range of 100 nm (Axsun Technologies, Inc.) as the source engine operating at a 50 kHz repetition rate with an axial resolution of 19 μm. To boost the computing performance, graphics processing unit (GPU) was utilized for regular OCT signal processing and three-dimensional visualization. The volume size is 512*512*512 pixels. The results are demonstrated in FIG. 4A-4B, which indicates the canal with a width of 770 μm.

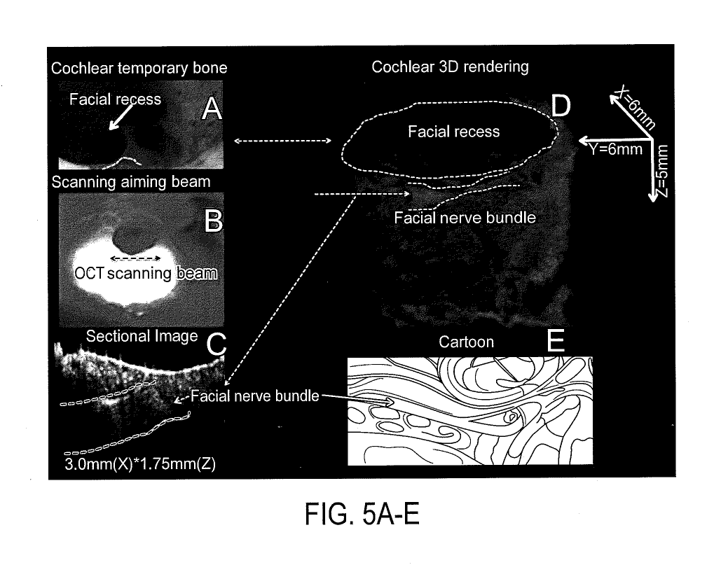

To identify the facial nerve bundle as well as other tissue structure in temporal bone, we perform a full three dimensional scan of a fresh human thinned cadaveric temporal bone over a range of 6 mm×6 mm×5 mm. FIGS. 5A-5E illustrate the results. The facial nerve bundle was clearly in a form of band structure in FIG. 5D and which can be also double confirmed within the dash line marked area in FIG. 5C.

To conclude, we have designed and demonstrated an OCT system according to an embodiment of the current invention that is capable of both CP-OCT single-mode fiber sapphire ball-lensed probe and bulk 3-D scanning of cochlea and surrounding temporal bone. A graphics processing unit (GPU) was used to boost the computing performance and to speed up the 3D volumetric rendering. To the best of our knowledge, both the basal turn and facial nerve bundles inside the human cochlear temporal bone were the first clearly identified with 2D and 3D OCT imaging systems. The OCT scanning head can also be used to image basilar membrane inside temporal bone. OCT guided cochlear implant surgery both works for spectral domain OCT (SDOCT) and swept source OCT and different wavelengths ranging from 850 nm, 1060 nm, 1310 nm to 1550 nm. The OCT scanning head can be attached to a robotic arm or other surgical tools and made of different portable sizes. OCT scanning guiding heads according to some embodiments of the current invention can be made with micro-electromechanical systems (MEMS) technology for microsurgery.

### REFERENCES

1. D. Huang, E. A. Swanson, C. P. Lin, J. S. Schuman, W. G. Stinson, W. Chang, M. R. Hee, T. Flotte, K. Gregory, C. A. Puliafito, and J. G. Fujimoto, “Optical coherence tomography,” Science 254, 4 (1991).
2. A. F. Fercher, C. K. Hitzenberger, G. Kamp, and S. Y. El-Zaiat, “Measurement of intraocular distances by backscattering spectral interferometry,” Opt. Commun. 17, 6 (1995).
3. M. Yamanari, S. Makita, Y. Lim, and Y. Yasuno, “Full-range polarization-sensitive swept-source optical coherence tomography by simultaneous transversal and spectral modulation,” Opt.Express 18, 17 (2010).
4. M. Zhao, A. N. Kuo, and J. A. Izatt1, “3D refraction correction and extraction of clinical parameters from spectral domain optical coherence tomography of the cornea,” Opt.Express 18, 14 (2010).
5. J. U. Kang, J.-H. Han, X. Liu, K. Zhang, C. G. Song, and P. Gehlbach, “Endoscopic Functional Fourier Domain Common-Path Optical Coherence Tomography for Microsurgery,” IEEE J. of Select. Topic in Quantum. Electron. 16, 12 (2010).
6. W. Chien, J. Niparko, I. Iordachita, R. Taylor, and J. Kang, “12th International Conference on Cochlear Implants and Other Implantable Auditory Technologies,” (2012).
7. G. J. Tearney, S. A. Boppart, B. E. Bouma, M. E. Brezinski, N. J. Weissman, J. F.

Southern, and J. G. Fujimoto, “Scanning single-mode fiber optic catheter-endoscope for optical coherence tomography,” Opt.Lett. 21, 3 (1996).

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. An optical coherence tomography (OCT) system for real-time surgical guidance, comprising:
an optical source;
an optical fiber configured to be optically coupled to said optical source;
a plurality of OCT sensor heads configured to be optically coupled to said optical fiber;
an optical detector configured to be optically coupled to said optical fiber;
a signal processor configured to communicate with said optical detector to receive detected signals therefrom; and
a display system configured to receive OCT image signals from said signal processor and to display an OCT image of at least a portion of a surgical region of interest in real time to provide surgical guidance,
wherein said plurality of OCT sensor heads comprises a bulk sensor head configured to image at least a portion of said surgical region of interest from an external imaging position and an endoscopic sensor head configured to be inserted into said surgical region of interest to image at least a portion of said surgical region of interest from an internal imaging position, and
wherein said bulk sensor head and said endoscopic sensor head are at least one of separate exchangeable sensor heads or a reconfigurable sensor head.

2. An OCT system according to claim 1, further comprising an optical rotary junction configured to be optically coupled to said optical fiber between said optical source and said plurality of OCT sensor heads.

3. An OCT system according to claim 1, wherein said signal processor comprises a graphics processor (GPU) programmed to accelerate processing of said detected signals to provide real-time OCT image signals.

4. An OCT system according to claim 3, wherein said GPU is programmed to provide real-time, three-dimensional OCT image signals.

5. An OCT system according to claim 1, wherein said bulk sensor head and said endoscopic sensor head are reconfigurable by attaching and removing an optical adapter to an end of said endoscopic sensor head.

6. An OCT system according to claim 1, wherein said bulk sensor head comprises a borescope having a size and used as an endoscope.

7. An OCT system according to claim 1, wherein said endoscopic sensor head comprises:
a sheath having a proximal end and a distal end, said sheath defining a lumen therein;
a sensor optical fiber disposed at least partially within said lumen of said sheath; and
a sapphire lens attached to said distal end of said sheath to form a fluid-tight seal to prevent fluid from entering said lumen of said sheath, and
wherein said sensor optical fiber has an end arranged in an optical path with said sapphire lens to provide optical coupling between said sapphire lens and said sensor optical fiber.

8. An OCT system according to claim 7, wherein said sapphire lens is a substantially spherical sapphire ball lens.

9. An OCT system according to claim 7, wherein said end of said sensor optical fiber is fixed within said lumen to maintain a predetermined distance from said sapphire lens with a space reserved therebetween.

10. An OCT system according to claim 1, wherein said bulk sensor head comprises a scanning mirror to scan illumination light, and to receive returned light, across a region to be imaged.

11. An OCT system according to claim 10, wherein said scanning mirror is a galvano mirror.

12. An OCT system according to claim 10, wherein said scanning mirror is a micro-electromechanical system (MEMS) mirror.

13. An OCT system according to claim 1, wherein said surgical region of interest is a cochlea region and said OCT system is configured to provide real-time guidance for cochlear implant surgery.

14. A method of performing a procedure using real-time OCT guidance, comprising:
obtaining an external OCT image of a region of interest with a bulk sensor head from an external position;
at least one of exchanging or reconfiguring said bulk sensor head with an endoscopic senor head;
obtaining an internal OCT image of at least a portion of region of interest with said endoscopic sensor head from an internal position; and
performing a step of said procedure taking into account information from at least one of said external or internal OCT images.

15. A method of performing a procedure according to claim 14, wherein said at least one of exchanging or reconfiguring said bulk sensor head with an endoscopic senor head maintains alignment with said region of interest.

16. A method of performing a procedure according to claim 14, wherein said procedure is a cochlear implant procedure.
